(19) United States
(12) Patent Application Publication
(10) Pub. No.: US 2016/0152656 A1
(43) Pub. Date: Jun. 2, 2016

(54) Methods of Using Phosphoramidation Reaction for Conjugation-Labeling Nucleic Acids

(71) Applicant: Kaohsiung Medical University, Kaohsiung City (TW)

(72) Inventors: Tzu-Pin Wang, Tainan City (TW); Yu-Chih Su, Kaohsiung City (TW)

(21) Appl. No.: 14/555,770

(22) Filed: Nov. 28, 2014

(51) Int. Cl. C07H 21/04

(52) U.S. Cl. CPC C07H 21/04

(57) ABSTRACT

Methods are provided to label nucleic acids through two-step phosphoramidation reactions with regiospecificity and high sensitivity. The methods can be widely used for synthesizing conjugates of nucleic acids. Through the two-step phosphoramidation reactions, nucleic acids are directly or indirectly labeled for easier and more efficient applications. The present invention also uses fluorescence quenching and DNA melting profile studies to demonstrate that the nucleic acids modified through the two-step phosphoramidation reactions preserve intrinsic base-pairing specificity and bestow regioselectivity in nucleic acids with high yield. Thus, the present invention has significant contribution to basic nucleic acid research; and, in the future, can be applied to effectively and quantitatively detect and measure nucleic acids inside and outside living organisms.

### FIELD OF THE INVENTION

The present invention relates to synthesizing labeling nucleic acids, and, more specifically, relates to easily synthesizing nucleic acids at the 5′ end with label molecules through covalent bondings, where the nucleic acids are directly or indirectly modified with diverse label molecules.

### BACKGROUND OF THE INVENTION

Labeling nucleic acids at a specific structural position has been widely used nowadays to detect and analyze specific nucleic acids. As a consequence, related techniques have played an essential role in scientific research and clinical applications. However, there is a big challenge for nucleic acid labeling methods, which is to more conveniently and cost-effectively introduce a label molecule to a natural or nonnatural nucleic acid without changing its base-pairing specificity for effectively detecting and quantifying the nucleic acid.

One of the commonly used nucleic acid labeling methods is done by enzyme catalysis. But this method must use expensive enzymes to render cost relatively high and not suitable for large scale analysis of nucleic acids. Another popular nucleic acid labeling method is done by solid-phase organic synthesis based on phosphoramidite chemistry. But this method inherits the intrinsic shortcomings of phosphoramidite chemistry with limits on synthesis and labeling of long nucleic acids.

Nucleic acid labeling method has been widely used in basic research and biotechnology industries, but, concerning modification of natural labeling nucleic acids, cost-effective and efficient methods for modifying nucleic acids are not developed. Therefore, high-performance methods for labeling natural DNAs/RNAs are important topics of research and applications. In addition, the development of novel and effective methods of labeling nucleic acids must be able to be applied to nucleic acids obtained either through enzyme synthesis or solid-phase organic synthesis. At the same time, reaction specificity must be achieved with label molecules efficiently modified on the nucleic acids; and the original base-pairing specificity of the nucleic acids should be preserved without using expensive enzymes. With all the aboves, labeled nucleic acids can be efficiently synthesized for expression analysis of common gene and quantitative detection of nucleic acids.

Hence, the prior arts do not fulfill all users' requests on actual use.

### SUMMARY OF THE INVENTION

The main purpose of the present invention is to provide a method based on a two-step phosphoramidation reaction to easily label nucleic acids with fluorophores.

Another purpose of the present invention is to provide a method to covalently link label molecules to the 5′ end of any natural or synthetic DNA or RNA having variable lengths.

Another purpose of the present invention is to provide an effective approach to link a label molecule such as biotin, a fluorophore or other nucleophilic molecule to a DNA or RNA molecule for labeling and analyzing a nucleic acid.

To achieve the above purpose, the present invention is methods for modifying nucleic acids with label molecules through a phosphoramidation reaction, comprising steps of (a) dissolving a nucleic acid and an activating reagent in a first buffer to be reacted for 70˜100 minutes (min) and obtaining a solution; (b) processing purification and precipitation with ethanol to obtain an intermediate product; and (c) dissolving the intermediate product in a second buffer to be added with a nucleophilic reagent to be reacted for 2.5˜3 hours (hr) for obtaining a final product, wherein the second buffer contains ethylene-diaminetetraacetic acid (EDTA); wherein the nucleophilic reagent is a fluorophore; wherein the fluorophore is a fluorescent molecule containing amine such as lissamine rhodamine B ethylenediamine (LRBE) or 5-(2-aminoethylamino)-1-naphthalenesulfonic acid sodium salt (EDANS); and wherein the method offers strategies to modify nucleic acids with label molecules through direct and indirect couplings. Accordingly, novel methods for modifying nucleic acids with label molecules through a phosphoramidation reaction are obtained.

### BRIEF DESCRIPTION OF THE DRAWINGS

The present invention will be better understood by the following detailed descriptions of the preferred embodiments according to the invention, taken in conjunction with the accompanying drawings, in which

FIG. 1 is the schematic view showing the preferred embodiments according to the present invention;

FIG. 2 is the schematic view showing the direct modification of the phosphate by the label molecule at the 5′ end of nucleic acid through the two-step phosphoramidation reaction;

FIG. 3 is the schematic view showing the indirect modification of the phosphate by the label molecule at the 5′ end of nucleic acid through the two-step phosphoramidation reaction;

FIG. 4 is the view showing the electrophoretic analysis results;

FIG. 5 is the view showing the UV melting curves of the labeled DNA duplexes; and

FIG. 6 is the view showing the fluorescence quenching of the labeled DNAs.

### DESCRIPTIONS OF THE PREFERRED EMBODIMENTS

The following description of the preferred embodiments is provided to understand the features and the structures of the present invention.

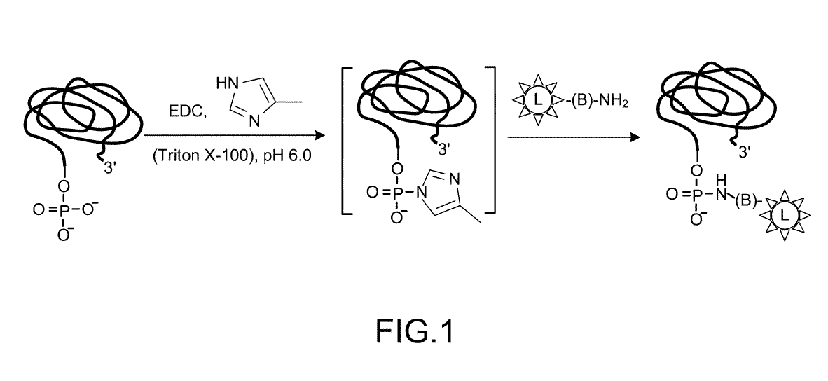

Please refer to FIG. 1 to FIG. 3, which are a schematic view showing preferred embodiments according to the present invention; and schematic views showing direct and indirect modifications of phosphate by a label molecule at the 5′ end of nucleic acids through a two-step phosphoramidation reaction. As shown in the figures, the present invention is methods for modifying nucleic acids with label molecules through a phosphoramidation reaction, where labeled nucleic acids are synthesized. The nucleic acids are easily modified with label molecules at the 5′ end in an aqueous solution without participation of enzymes. The synthesis methods are two-step phosphoramidation reactions to modify nucleic acids with diverse label molecules at the 5′ end through direct coupling or indirect coupling, where only a small equivalent number of label molecules is required. In FIG. 1, ‘L’ indicates a label molecule such as a biotin, a fluorophore or a radioisotope probe. The structure within the bracket in FIG. 1 is a reaction intermediate of phosphorimidazolide, where the reaction intermediate is not purified in the one-step phosphoramidation reaction, but is purified in the two-step phosphoramidation reaction prior to be reacted with a nucleophilic reagent. Besides, ‘B’ indicates a bridging molecule (i.e., a crosslinker), which is only required for indirectly labeling nucleic acids. Thus, the present invention uses a novel and simple phosphoramidation reaction to efficiently produce modified nucleic acids which are conjugated with label molecules such as fluorophores. The fluorophores are commercially available fluorescent molecules for biochemistry and molecular biology, including Lissamine rhodamine B ethylenediamine (LRBE), fluorescein Isothiocyanate (FITC), and 5-(2-aminoethylamino)-1-naphthalenesulfonic acid sodium salt (EDANS). The present invention is expected to effectively and economically modify unlimited lengths of natural or synthetic fragments of DNA or RNA with label molecules for detecting and analyzing nucleic acids, where a high yield can be achieved in a short period of time and common laboratory setups are enough to synthesize modified nucleic acids for basic scientific research, biotechnology developments and commercial production applications.

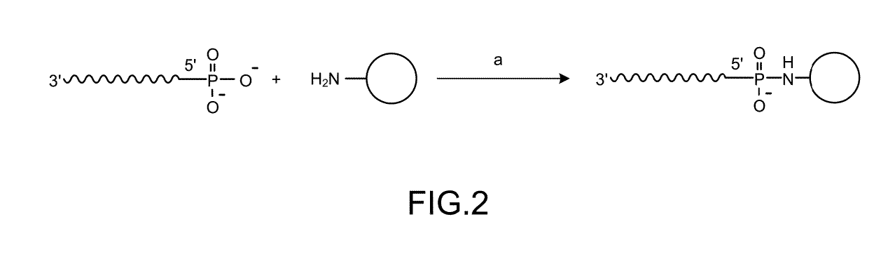

The method for direct modifications of nucleic acids as shown in FIG. 2 comprises the following steps:

(a1) A nucleic acid having phosphate at the 5′ end is dissolved together with an activating reagent in a first buffer for reaction for 70˜100 minutes (min). Therein, the activating reagent is N-(3-dimethylaminopropyl)-N′-ethylcarbodiimide hydrochloride (EDC); and, the first buffer is a 4(5)-methylimidazole solution having a pH value of 5˜6 and a concentration of 0.08˜0.12 M.

(b1) Purification and precipitation with ethanol are processed to a solution formed in step (a1) to acquire an intermediate product, 5′-phosphorimidazolide.

(c1) The intermediate product is dissolved in a second buffer containing ethylenediaminetetraacetic acid (EDTA) to be added with a nucleophilic reagent for reaction for 2.5˜3 hours (hr) to form a final product. Therein, the second buffer is a N-(2-hydroxyethyl)piperazine-N′-(3-propanesulfonic acid) (EPPS) solution having a pH value of 7˜8; the nucleophilic reagent is a biotin, a fluorophore or a radioisotope probe which contains a nucleophilic functional group; and, the fluorophore is a fluorescent molecule containing amine, like LRBE and EDANS.

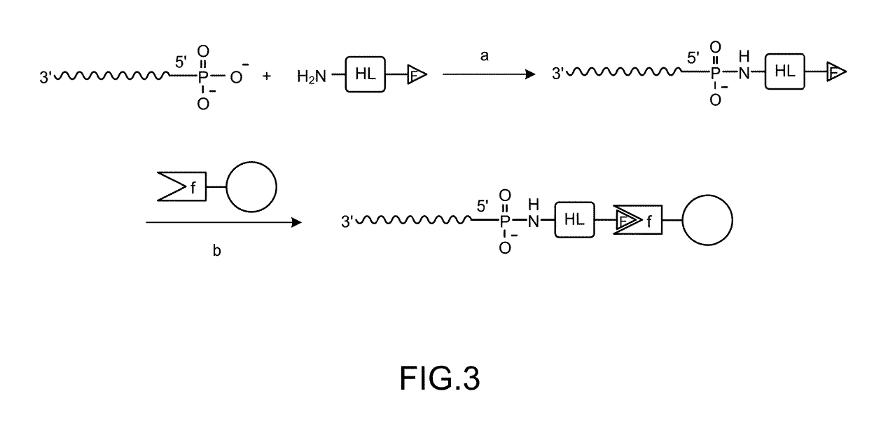

The method for indirect modifications of nucleic acids as shown in FIG. 3 comprises the following steps:

(a2) A nucleic acid having phosphate at the 5′ end is dissolved together with an activating reagent in a first buffer for reaction for 70˜100 min. Therein, the activating reagent is EDC; and, the first buffer is a 4(5)-methylimidazole solution having a pH value of 5˜6 and a concentration of 0.08˜0.12 M.

(b2) Purification and precipitation with ethanol are processed to a solution formed in step (a2) to acquire an intermediate product, 5′-phosphorimidazolide.

(c2) The intermediate product is dissolved in a second buffer containing EDTA to be added with a nucleophilic crosslinker for reaction for 2.5˜3 hrs. Therein, the second buffer is an EPPS solution having a pH value of 7˜8; and, the nucleophilic crosslinker is a homobifunctional (e.g. diamino) crosslinker (e.g. ethylenediamine) or a heterobifunctional crosslinker.

(d2) Purification and precipitation with ethanol are processed to a solution formed in step (c2) to form a conjugate of the nucleic acid modified with ethylenediamine.

(e2) The conjugate of the nucleic acid modified with the nucleophilic crosslinker is dissolved in a third buffer to be added with a labeling reagent for reaction for 05˜12 hrs to form a final product. Therein, the third buffer can be a sodium carbonate (Na₂CO₃) solution having a pH value of 8˜9 and a concentration of 0.08˜0.12 M; and, the labeling reagent is a biotin, a fluorophore (e.g. FITC), or a radioisotope probe.

In FIG. 2 and FIG. 3, ‘a’ is a two-step phosphoramidation reaction; in FIG. 3, ‘b’ is a reaction of modifying a nucleic acid having functional group F with a label molecule having the functional group f; and ‘HL’ is a homobifunctional or heterobifunctional cross-linker. Thus, a novel method for modifying nucleic acids with label molecules through a phosphoramidation reaction is obtained.

On using the present invention, a nucleic acid can be directly modified with a fluorophore by the following procedure:

1.59 nanomoles (nmol) of a single-strand DNA is dissolved in 20 microliters (μL) of a 4(5)-methylimidazole buffer [0.1 molar (M), pH 6.0] containing 20.87 micromoles (μmol) of EDC for reaction at a room temperature for 90 min. Then, ethanol is used for precipitation and purification to obtain an intermediate product, 5′-phosphorimidazolide. Then, 5′-phosphorimidazolide is dissolved in 27.5 μL of an EPPS buffer (300 mM EPPS, pH 8.0) containing 5 millimolars (mM) of EDTA to be added with 5 μL of 187.2 mM EDANS or LRBE dissolved in DMF as a nucleophilic reagent for reaction in the dark for 3 hrs at 55 Celsius degrees (° C.). A final product is thus obtained to be precipitated and purified with ethanol. Then, the final product is analyzed with 20 percents (%) of urea-polyacrylamide-gel-electrophoresis (urea-PAGE). The electrophoretic analysis result is processed through quantitative analysis by using Amersham Typhoon Phosphorlmager for calculating a yield.

On the other hand, on using the present invention, a nucleic acid can be indirectly modified with a fluorophore by the following procedure:

1.59 nmol of a single-strand DNA is dissolved in 20 μL of a 4(5)-methylimidazole buffer (0.1 M, pH 6.0) containing 20.87 μmol of EDC for reaction at a room temperature for 90 min. Then, ethanol is used for precipitation and purification to obtain an intermediate product, 5′-phosphorimidazolide. Then, 5′-phosphorimidazolide is dissolved in 27.5 μL of an EPPS buffer (300 mM EPPS, pH 8.0) containing 5 mM of EDTA. 5 μL of a 187.2 mM ethylenediamine solution is added for reaction at 55° C. for 3 hrs. Then, ethanol is used for precipitation and purification to obtain a conjugate of DNA modified with ethylenediamine. The conjugate of DNA modified with ethylenediamine is dissolved in 20 μL of a sodium carbonate buffer (0.1 M, pH 9.0) to be added with 20 μL of 2.57 mM FITC (dissolved in DMSO) as an electrophilic agent for reaction for 12 hrs in the dark at a room temperature. After purification and precipitation with ethanol, a final product is obtained, which is the DNA labeled with FITC. At last, a gel is used for purification to remove extra fluorescent dye for analysis with 20% of urea-PAGE. The electrophoretic analysis result is processed through quantitative analysis by using Amersham Typhoon Phosphorlmager for calculating a yield.

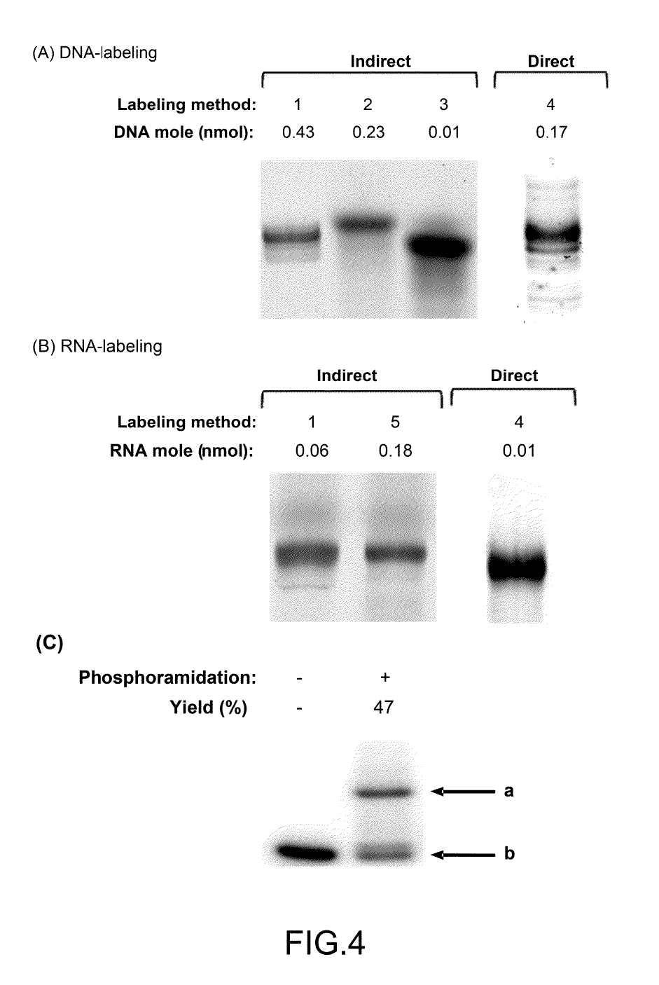

Please refer to FIG. 4, which is a view showing the electrophoretic analysis results. As shown in the figure, efficiencies of sample (A) and sample (B) are compared after labeling a DNAs and an RNA with fluorophores through a two-step phosphoramidation reaction or traditional nucleic-acid-labeling methods. Therein, sample (A) is a DNA labeled with fluorophores, electrophoretically analyzed with 20% urea-PAGE, and visualized through Amersham Typhoon Phosphorlmager. Sample (B) is an RNA labeled with fluorophores, electrophoretically analyzed with 8% urea-PAGE, and visualized through Amersham Typhoon Phosphorlmager. Sample (C) is a ³²P-labelled DNA specifically and selectively labeled with LRBE through the two-step phosphoramidation reaction. The DNA labeled with LRBE are precipitated with ethanol, analyzed with 20% urea-PAGE and, then, visualized and quantified by Amersham Typhoon Phosphorlmager. Therein, ‘a’ is the DNA labeled with LRBE; and, ‘b’ is the DNA unreacted.

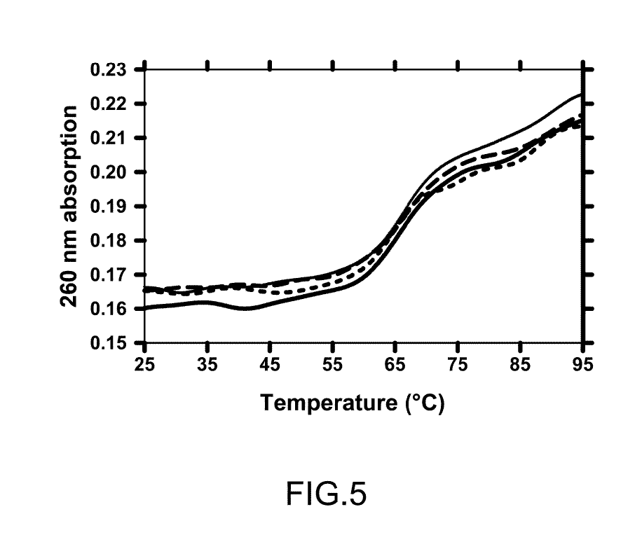

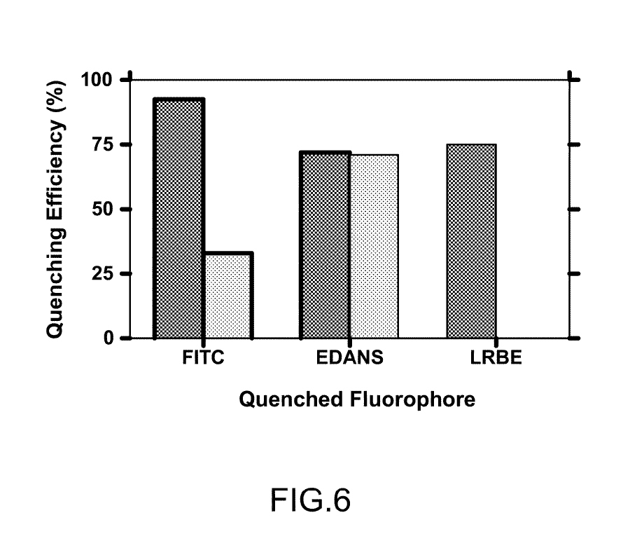

Please refer to FIG. 5 and FIG. 6, which are a view showing ultraviolet (UV) melting curves of labeled DNA duplexes; and a view showing fluorescence quenching of the labeled DNAs. As shown in the figures, integrity and hybridization specificity of the fluorescently-labeled nucleic acids synthesized according to the present invention are confirmed. In FIG. 5, DNA duplexes are used as samples for detecting their UV melting curves, where the detections are processed under concentrations of 0.4˜0.6 μM and each has a total volume of 800 μL. Both fluorescently-labeled single-strand and sequence-complemented single-strand DNA molecules are dissolved in a hybridization buffer and heated to 65° C. for 5 min, then kept at a room temperature for 1 hour and finally transferred to a 1-mL quartz cuvette. The cuvette is sealed with a cap and heated up from 25° C. to 95° C. at a speed of 0.5° C. per minute by using a computer for controlling the temperature, where changes of 260 nm absorption (A₂₆₀) are recorded at each temperature to form curve graphs and obtain Tₘ values. Among the UV thermal melting curves of the DNA duplexes shown in FIG. 5, the black solid curve is for the original single-strand DNA; the black broken curve is for the single-strand DNA labeled with FITC; the black dotted curve is for the single-strand DNA labeled with LRBE; and the bold black solid curve is for the single-strand DNA labeled with EDANS. The results demonstrated that the two-step phosphoramidation reactions in the present invention efficiently labels nucleic acids with fluorophores (such as LRBE, FITC or EDANS) without affecting the hybridization specificity in the nucleic acids.

Furthermore, in a fluorescence-quenching experiment, the single-strand DNA labeled with fluorophores at the 5′ end is dissolved in a hybridization buffer (20 mM of Tris-HCl, 5 mM of MgCl₂ and 50 mM of NaCl, pH 7.5) to obtain 3 μM nucleic acid solutions (each having a total volume of 200 μL). According to the excitation and emission wavelengths of the fluorophores, the fluorescence of the single-strand DNAs labeled with fluorophores at the 5′ end is detected. Then, the single-strand DNAs labeled with fluorophores at the 5′ end are hybridized with sequence-complemented single-strand DNAs labeled with DABCYL [4-(dimethylaminoazo)benzene-4-carboxyl] at the 5′ or 3′ end. The single-strand DNA containing DABCYL has a final concentration of 15 μM. After being mixed altogether, the DNAs are heated up at 70° C. for 5 min and then cooled down at room temperature for 30 min for detecting fluorescence and calculating fluorescence quenching in the end. In FIG. 6, the regioselectivity of labeling a nucleic acid at the 5′ end with a fluorophore through the two-step phosphoramidation reactions is supported by the results of fluorescence resonance energy transfer (FRET) quenching and contact quenching. It is noted that the DNAs conjugated with EDANS, FITC and LRBE are detected by their fluorescences at 473 nm, 520 nm and 590 nm, respectively.

The present invention uses the aqueous-phase two-step phosphoramidation reactions to modify nucleic acids with label molecules at the 5′ end, which comprises two strategies:

(a) Phosphate groups at the 5′ end of nucleic acids are directly modified with label molecules to form intermediate products phosphorimidazolide where the intermediate products can be directly reacted with the nucleophilic fluorophores (e.g. LRBE, EDANS or other fluorescent amine-containing compounds) to form nucleic acid conjugates.

(b) Homobifunctional (e.g. amino) or heterobifunctional crosslinkers are used to form conjugates to link nucleic acids with fluorophores (such as FITC) afterwards.

Thus, the present invention successfully directly modifies nucleic acids with EDANS and LRBE; or, indirectly links FITC at the 5′ end of nucleic acids by using ethylenediamine as a crosslinker. The fluorescent labeled nucleic acid products thus obtained are used for analyzing nucleic acids by FRET and contact quenching, which demonstrates that the present invention has regioselectivity on modifying nucleic acids with label molecules at the 5′ end. In addition, through determination of nucleic acid melting temperatures, the above conjugates of nucleic acids are shown to have the same hybridization specificity as the original nucleic acids.

Hence, the labeling methods for nucleic acids in the present invention can easily conjugate nucleic acids with other label molecules having multiple functional groups with short reaction time in good yields, which are suitable for labeling DNA or RNA molecules to obtain a variety of labeled nucleic acids. In addition, the present invention also preserves the base-pairing specificity of the nucleic acids, so that the labeled nucleic acids can unequivocally, effectively and quantitatively detect and measure nucleic acids essential to biotechnology and pharmaceutical industries. The fluorescent labeled nucleic acids can be used as tools for molecular biological developments and clinical applications. The conjugates of nucleic acids paired by fluorescent dye-quencher can be applied for basic research including FRET and nucleic acid sensor developments in the future. The product applications may include derivatives of chemically-labeled nucleic acid molecules: By using the present invention, label molecules, like a biotin or a fluorophores, can be effectively linked to DNA and RNA molecules for detecting and analyzing nucleic acids.

To sum up, the present invention is methods of modifying nucleic acids with label molecules through a phosphoramidation reaction, where, the present invention is to label nucleic acids based on a two-step phosphoramidation reaction; any length of a natural or synthetic DNA or RNA can be processed through a covalent linkage modification at the 5′ end of nucleic acids; and, thus, the present invention can be applied for synthesizing conjugates of fluorescent labeled nucleic acids.

The preferred embodiments herein disclosed are not intended to unnecessarily limit the scope of the invention. Therefore, simple modifications or variations belonging to the equivalent of the scope of the instructions disclosed herein for a patent are all within the scope of the present invention.

What is claimed is:

1. A method of modifying a nucleic acid with a label molecule through a phosphoramidation reaction, the method modifying said label molecules on said nucleic acids through direct coupling and comprising steps of:
(a1) dissolving a nucleic acid and an activating reagent in a first buffer to be reacted for 70˜100 minutes (min) to obtain a solution, said nucleic acids having phosphate at the 5′ end;
(b1) processing purification and precipitation with ethanol to obtain an intermediate product; and
(c1) dissolving said intermediate product in a second buffer to be added with a nucleophilic reagent to be reacted for 2.5˜3 hours (hr) to obtain a final product,
wherein said second buffer contains ethylene-diaminetetraacetic acid (EDTA);
wherein said nucleophilic reagent is a fluorophore; said fluorophore is a fluorescent molecule containing amine; and said fluorophore is selected from a group consisting of lissamine rhodamine B ethylenediamine (LRBE) and 5-(2-aminoethylamino)-1-naphthalenesulfonic acid sodium salt (EDANS).

2. The method according to claim 1,
wherein said nucleic acids is a length of a material selected from a group consisting of a DNA and an RNA; and
wherein said material is selected from a group consisting of a natural material and a synthetic material.

3. The method according to claim 1,
wherein said activating reagent is N-(3-dimethylaminopropyl)-N′-ethylcarbodiimide hydrochloride (EDC).

4. The method according to claim 1,
wherein said first buffer is a 4(5)-methylimidazole solution and has a pH value of 5˜6 and a concentration of 0.08˜0.12 molars (M).

5. The method according to claim 1,
wherein said intermediate product is 5′-phosphorimidazolide.

6. The method according to claim 1,
wherein said second buffer is a N-(2-hydroxyethyl)piperazine-N′-(3-propanesulfonic acid) (EPPS) solution and has a pH value of 7˜8.

7. The method according to claim 1,
wherein said nucleophilic reagent is selected from a group consisting of a biotin, a fluorophore and a radioisotope probe and said nucleophilic reagent contains at least one nucleophilic functional group.

8. A method of modifying a nucleic acid with a label molecule through a phosphoramidation reaction, the method modifying said label molecules on said nucleic acids through indirect coupling and comprising steps of:
(a2) dissolving a nucleic acid and an activating reagent in a first buffer to be reacted for 70˜100 min, said nucleic acid having phosphate at the 5′ end;
(b2) processing purification and precipitation with ethanol to obtain an intermediate product;
(c2) dissolving said intermediate product in a second buffer to be added with a nucleophilic crosslinker to be reacted for 2.5˜3 hrs, said second buffer containing EDTA;
(d2) processing precipitation with ethanol and processing purification with a urea-PAGE gel to obtain a conjugate of said nucleic acids modified with said nucleophilic crosslinkers; and
(e2) dissolving said conjugates of said nucleic acids in a third buffer to be added with a labeling reagent to be reacted for 0.5˜12 hrs to obtain a final product.

9. The method according to claim 8,
wherein said nucleic acids is a length of a material selected from a group consisting of a DNA and an RNA; and
wherein said material is selected from a group consisting of a natural material and a synthetic material.

10. The method according to claim 8,
wherein said activating reagent is EDC.

11. The method according to claim 8,
wherein said first buffer is a 4(5)-methylimidazole solution and has a pH value of 5˜6 and a concentration of 0.08˜0.12 M.

12. The method according to claim 8,
wherein said intermediate product is 5′-phosphorimidazolide.

13. The method according to claim 8,
wherein said second buffer is an EPPS solution and has a pH value of 7˜8.

14. The method according to claim 8,
wherein said nucleophilic crosslinker is selected from a group consisting of a homobifunctional crosslinker and a heterobifunctional crosslinker.

15. The method according to claim 14,
wherein said the homobifunctional crosslinker is ethylenediamine.

16. The method according to claim 8,
wherein said conjugates of said nucleic acids are nucleic acids modified with ethylenediamine.

17. The method according to claim 8,
wherein said the third buffer is a sodium carbonate (Na2CO3) solution and has a pH value of 8˜9 and a concentration of 0.08˜0.12 M.

18. The method according to claim 8,
wherein, in step (e), said a labeling reagent is selected from a nucleophilic reagent and a electrophilic agent and is selected from a group consisting of
a group consisting of a biotin, a fluorophore and a radioisotope probe.

19. The method according to claim 18,
wherein said a fluorophore is a fluorescent molecule containing amine and is selected from a group consisting of LRBE, EDANS and an electrophilic fluorophore.

20. The method according to claim 19,
wherein said an electrophilic fluorophore is fluorescein isothiocyanate (FITC).
